(19) United States
(12) Patent Application Publication
(10) Pub. No.: US 2024/0270781 A1
(43) Pub. Date: Aug. 15, 2024

(54) PREPARATION METHOD OF PAEONIFLORIN-6'-O-BENZENE SULFONATE

(71) Applicant: GUANGZHOU HANFANG PHARMACEUTICAL CO., LTD., Guangzhou, Guangdong (CN)

(72) Inventors: Wendong XU, Guangzhou, Guangdong (CN); Cheng YUAN, Guangzhou, Guangdong (CN); Shunzhi TANG, Guangzhou, Guangdong (CN); Juyan LIU, Guangzhou, Guangdong (CN); Hongfei CAI, Guangzhou, Guangdong (CN); Bai BAI, Guangzhou, Guangdong (CN); Jing LI, Guangzhou, Guangdong (CN); Yuping FU, Guangzhou, Guangdong (CN); Songtao LI, Guangzhou, Guangdong (CN); Wancai PENG, Guangzhou, Guangdong (CN); Yonghua LI, Guangzhou, Guangdong (CN); Yao LI, Guangzhou, Guangdong (CN); Jirong LI, Guangzhou, Guangdong (CN); Shaoheng WEI, Guangzhou, Guangdong (CN); Jiali LI, Guangzhou, Guangdong (CN)

(21) Appl. No.: 18/290,641

(22) PCT Filed: Oct. 19, 2021
(86) PCT No.: PCT/CN2021/124536

§ 371 Date: Jan. 19, 2024

(30) Foreign Application Priority Data
Jul. 23, 2021 (CN) 202110838326.1

(51) Int. Cl. C07H 17/04

(52) U.S. Cl. CPC C07H 17/04

(57) ABSTRACT

Provided in the present disclosure is a preparation method of paeoniflorin-6′-O-benzene sulfonate. The method uses crude paeoniflorin instead of paeoniflorin as a raw material, which more complies with actual requirements of an industrial production process of paeoniflorin-6′-O-benzene sulfonate, and uses an organometallic catalyst, an acid-binding agent and chemically active benzenesulfonyl chloride to react with paeoniflorin. Compared with the prior art, the method significantly increases the yield of crude paeoniflorin-6′-O-benzene sulfonate which is separated and purified by means of column chromatography and then refined to obtain paeoniflorin-6′-O-benzenesulfonate with a content of at least 98%, thereby realizing industrial production of the paeoniflorin-6′-O-benzene sulfonate.

### CROSS-REFERENCE TO RELATED APPLICATIONS

The present application is a national phase entry under 35 USC § 371 of International Application PCT/CN2021/124536, filed Oct. 19, 2021, which claims the benefit of and priority to Chinese Patent Application No. 202110838326.1, filed Jul. 23, 2021. The entire disclosures of the above applications are incorporated herein by reference.

### TECHNICAL FIELD

The present disclosure belongs to the technical field of compound preparation, and particularly relates to a preparation method of paeoniflorin-6′-O-benzene sulfonate.

### BACKGROUND

Radix Paeoniae Lactiflorae is the root of paeonia lactiflora, which has antipyretic and analgesic effects. Modern pharmacological research has shown that a Radix Paeoniae Lactiflorae extract also has various pharmacological activities such as anti-inflammatory, immunoregulation, detoxification, liver protective, anti-mutagenic, anti-tumor effects. Total glucoside of paeony is also the first anti-inflammatory and immunomodulatory drug for clinically treating rheumatoid arthritis with the characteristics including good curative effect, few adverse reactions, good tolerance and the like. Paeoniflorin (Pae) is considered as the main effective component of the total glucoside of paeony, and is often used as the main active ingredient of other traditional Chinese medicine compound preparations for clinical treatment. It is demonstrated that Pae has remarkable pharmacological effects, but its low oral bioavailability and slow onset bring difficulties to its drug promotion. In order to solve this problem, Wei et al. conducted a series of studies on Pae, and Pae was structurally modified via derivatization. A series of compounds were synthesized from Pae, wherein a preferred derivative, paeoniflorin-6′-O-benzene sulfonate, showed good pharmaceutical activity.

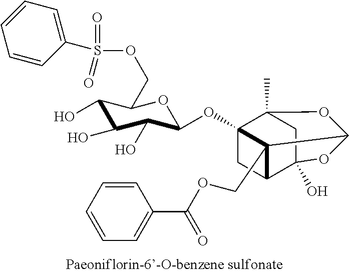

Furthermore, it was found in an animal experiment that paeoniflorin-6′-O-benzene sulfonate showed better drug absorption and distribution in animals than those of Pae, had a slower rate of drug metabolism, and showed better bioavailability. In a further pharmacological study, the strong immunomodulatory function of paeoniflorin-6′-O-benzene sulfonate had been found. Chen found that paeoniflorin-6′-O-benzene sulfonate had a good therapeutic effect on primary Sjögren's syndrome by inhibiting the regulation of the upper GRK2-JAK1-STAT1/2-CXCL13 signaling in submandibular gland epithelial cells. Good pharmacological activity and fewer adverse reaction laid a solid foundation for the drug development of paeoniflorin-6′-O-benzene sulfonate.

In the prior art, the earliest reported preparation method of paeoniflorin-6′-O-benzene sulfonate was that Pae with a content of no less than 98% was taken as a raw material, dissolved in 20-80 times parts by weight thereof of an organic solvent. In the presence of a catalyst in an amount of 10%-15% by mass of paeoniforin, halogenated hydrocarbon in an amount of 50-100 fold by volume of acylating agent was added dropwise to synthesize paeoniflorin-6′-O-benzene sulfonate with a yield of only about 20%. Later, some scholars improved the process. With Pae as the raw material, pyridine (1.3 mL/g) as an acid binding agent and chloroform (6.6 mL/g) as a reaction solvent, the reaction mixture was stirred at constant temperature for a period of time; and then 2-fold benzenesulfonyl chloride by molar ratio of paeoniflorin was slowly added dropwise within at least 4 hours. Under the condition of reaction time of 10 hours, the yield reached about 58%.

However, the existing synthesis method of paeoniflorin-6′-O-benzene sulfonate has the following defects: 1. The required content of Pae as the reaction raw material of no less than 98% results in a restriction in source of raw materials, and there is no kilogram-grade raw material with a content equal to or above 98% in the market at present. 2. When paeoniflorin with a content of 98% is used as a raw material, a yield of a product is low, below 60%; and the reaction process is uneconomical and environmentally friendly. 3. Large amount of the catalyst and reaction solvent are required in the reaction. 4. The reaction raw materials need to be added stepwise and slowly added dropwise in the reaction process, a long reaction time is required to prepare products on a large scale, and the process is complicated, which are not conducive to industrial production. Therefore, an industrial synthesis and purification method for preparing paeoniflorin-6′-O-benzene sulfonate on a large scale is imminent for pre-clinical research and drug marketing preparation.

### SUMMARY

The present disclosure aims at solving at least one of the above-mentioned technical problems in the prior art. Therefore, a first aspect of the present disclosure provides a preparation method of paeoniflorin-6′-O)-benzene sulfonate, which can realize industrial production of paeoniflorin-6′-O-benzene sulfonate.

The present disclosure provides a preparation method of paeoniflorin-6′-O-benzene sulfonate with a reaction route as follows:

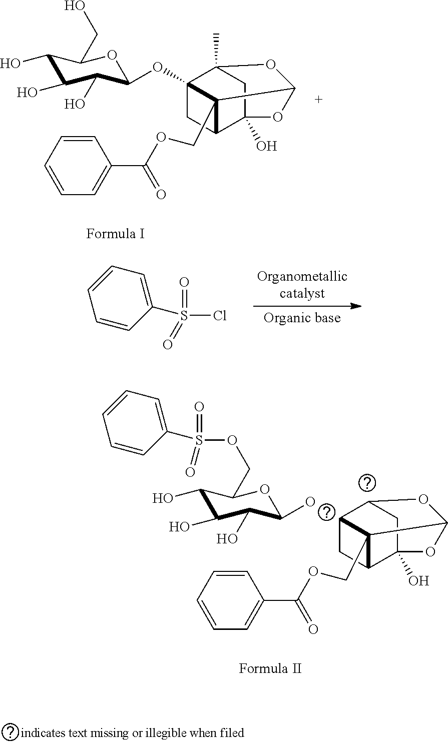

Specifically, the preparation method comprises the following steps of:
S1: adding an organic solvent, an organometallic catalyst, an acid binding agent and benzenesulfonyl chloride into paeoniflorin for reaction, and concentrating a reaction product after the reaction is terminated to obtain a crude product of paeoniflorin-6′-O-benzene sulfonate;
S2: separating the crude product of paeoniflorin-6′-O-benzene sulfonate by column chromatography, and collecting an eluate to obtain paeoniflorin-6′-O-benzene sulfonate with a content of at least 95%;
S3: recrystallizing paeoniflorin-6′-O-benzene sulfonate obtained in step S2 in a mixed solvent to obtain paeoniflorin-6′-O-benzene sulfonate with a content of at least 98%.

According to the method provided by the present disclosure, in step S1, the organometallic catalyst, the acid binding agent and the benzenesulfonyl chloride with active chemical properties are reacted with paeoniflorin; so that a reaction yield can reach more than 75%, and a yield of the crude product of paeoniflorin-6′-O-benzene sulfonate can be significantly improved. After separating and purifying the crude product of paeoniflorin-6′-O-benzene sulfonate by column chromatography, the paeoniflorin-6′-O-benzene sulfonate with a content of more than 98% can be obtained.

In some preferred embodiments of the present disclosure, before step S1, the method further comprises a step of separating a crude product of paeoniflorin by column chromatography to obtain paeoniflorin with a content of at least 85%.

Preferably, the crude product of paeoniflorin is derived from a natural plant extract, for example, may be derived from a water extract and alcohol precipitate of any one of plant such as Radix Paeoniae Lactiflorae, Radix Paeoniae Rubra, cortex moutan, rhizoma cibotii, peony root, and purple peony root.

Preferably, a content of paeoniflorin in the crude product of paeoniflorin is greater than 40%; more preferably, greater than 45%; further preferably, greater than 50%: particularly preferably, a content of paeoniflorin in the natural plant extract is greater than 55%, or further greater than 60%; most preferably, greater than 65%; most preferably, greater than 70%; particularly preferably, greater than 75%; and especially preferably, greater than 80%.

In the present disclosure, the crude product of paeoniflorin instead of high-content paeoniflorin is used as a raw material. The crude product of paeoniflorin is more in line with actual requirements of an industrial production process of paeoniflorin-6′-O-benzene sulfonate due to the fewer pre-treatment, simplicity of preparation, and a low cost (less than 0.1% of the price of paeoniflorin with a content of 98%).

In some more preferred embodiments of the present disclosure, during the purification of the crude product of paeoniflorin, an eluent for the column chromatography is a mixed solvent of alkane derivative and alcohol; and further preferably, a volume ratio of the alkane derivative to the alcohol is 3:1 to 15:1, and preferably 5:1 to 9:1.

Further preferably, the alkane derivative is ethyl acetate, isopropyl acetate, chloroform, dichloromethane, or a combination thereof.

Preferably, the alcohol is lower alcohol; and more preferably, is methanol, ethanol, isopropyl alcohol or a combination thereof.

Further preferably, the eluent for the column chromatography is a mixed solvent of ethyl acetate and methanol or a mixed solvent of ethyl acetate and ethanol.

Preferably, a mixed volume ratio of the ethyl acetate to the methanol is 3:1 to 7:1; and further preferably, is 4:1 to 6:1.

An alkane derivative-alcohol system is used for separating the crude product of paeoniflorin by column chromatography in the present disclosure. Compare with a haloalkane-alcohol system, the alkane derivative-alcohol system exhibits a stronger elution ability, such that less eluent is required to achieve a good separation effect. In addition, the toxicity of the alkane derivative-alcohol system is less than that of chloroform, so that the production is safer and more environment-friendly.

In some more preferred embodiments of the present disclosure, during the purification of the crude product of paeoniflorin, in the column chromatography, a volume-to-mass ratio of the eluent to the crude product of paeoniflorin is 35:1 mL/g to 55:1 mL/g; and preferably, 35:1 mL/g to 45:1 mL/g.

In some more preferred embodiments of the present disclosure, during the purification of the crude product of paeoniflorin, a filler in the column chromatography is silica gel; preferably, the silica gel has a 100 to 300 mesh size; and preferably, the silica gel has a 50 A to 90 A pore size.

A mass ratio of the silica gel to the crude product of paeoniflorin is 5:1 to 12:1; and further preferably, is 7:1 to 9:1.

In the present disclosure, more filler leads to a better separation effect; and when the filler is present in an amount of 7 to 9 times the weight of the crude product of paeoniflorin, less eluent is required on the premise of ensuring a better separation effect.

In some more preferred embodiments of the present disclosure, during the purification of the crude product of paeoniflorin, before the reaction, the method further comprises a step of carrying out, dissolution for preliminarily removing impurities, adsorption, elution and concentration on the crude product of paeoniflorin.

Further preferably, the crude product of paeoniflorin is dissolved with lower alcohol, the lower alcohol is methanol, ethanol, isopropyl alcohol or a combination thereof, and a volume-to-mass ratio of the lower alcohol to the crude product of paeoniflorin is preferably 1:1 mL/g to 3:1 mL/g.

Further preferably, the adsorption may be adsorption under stirring and/or column chromatography adsorption; further preferably, an adsorbent for the adsorption is selected from silica gel or aluminium oxide; further preferably, a mass ratio of the adsorbent to the crude product of paeoniflorin is 2:1 to 5:1; further preferably, an eluent for the elution is pure ethanol, and a volume-to-mass ratio of the eluent to the crude product of paeoniflorin is 4:1 mL/g to 6:1 mL/g.

Generally speaking, the crude product of paeoniflorin not only contains more than 40% by weight thereof of paeoniforin, but also contains a large amount of impurities such as tannins and carbohydrate gum. A direct separation by column chromatography will not only affects the content of paeoniflorin, but also has a great impact on the separation efficiency. Therefore, the content and separation efficiency of paeoniflorin can be further improved by the method of extracting the crude product of paeoniflorin from plants (such as Radix Paeoniae Lactiflorae and Radix Paeoniae Rubra) by the water extraction and alcohol precipitation method and subsequently removing the impurities (such as tannin and carbohydrate gum) from paeoniflorin by using the adsorbent.

The adsorbent used in the present disclosure, such as silica gel and aluminium oxide, has a low price and good separation effect, which can reduce the industrial production cost of paeoniflorin-6′-O-benzene sulfonate. In order to further improve the separation efficiency of paeoniflorin, the crude product of paeoniflorin can be dissolved in the same solvent as the eluent used in column chromatography before column chromatography.

In some more preferred embodiments of the present disclosure, the organometallic catalyst in step S1 is selected from the group consisting of C₁-C₄ alkyl tin halide, aryl tin halide, C₁-C₄ alkyl tin oxide and C₁-C₄ alkyl tin sulfide; further preferably, the organometallic catalyst in S1 is at least one selected from the group consisting of dimethyl tin dichloride, diethyl tin dichloride, di-tert-butyl tin dichloride, diphenyl tin dichloride, dimethyl tin oxide and dimethyl tin sulfide.

Further preferably, the organometallic catalyst is added in an amount of 0.035% to 45% by weight of paeoniflorin; further preferably, the organometallic catalyst is added in an amount of 0.5% to 10.0% by weight of paeoniflorin; and further preferably, the organometallic catalyst is added in an amount of 0.6% to 7.0% by weight of paeoniflorin.

In some more preferred embodiments of the present disclosure, the acid binding agent in step S1 is at least one selected from the group consisting of triethylamine, N,N′-diisopropylethylamine, pyridine, potassium carbonate, potassium bicarbonate, sodium carbonate, and sodium bicarbonate; further preferably, a molar ratio of paeoniflorin to the acid binding agent is 1:1 to 1:2; and further preferably, is 1:1.2 to 1:1.6.

Pyridine can be used as a reaction solvent for step S1 and an acid binding agent. However, due to the reproductive toxicity, pyridine is not the best choice for the industrial production of paeoniflorin-6′-O-benzene sulfonate, so it is not suitable for industrial application on a large scale.

In some more preferred embodiments of the present disclosure, the benzenesulfonyl chloride in step St is added in an amount of 20% to 60% by weight of paeoniflorin; and further preferably, the benzenesulfonyl chloride in S1 is added in an amount of 25% to 30% by weight of paeoniflorin.

In some more preferred embodiments of the present disclosure, the solvent for the reaction in step S1 is at least one selected from the group consisting of acetonitrile, tetrahydrofuran, acetone, pyridine, methyl ethyl ketone, N,N′-dimethylformamide, 1,4-dioxane and dimethyl sulfoxide; further preferably, a volume-to-mass ratio of the solvent to paeoniflorin is 2:1 mL/g to 10:1 mL/g.

In the present disclosure, benzenesulfonyl chloride with active chemical properties is used as a substrate, which needs to be used under anhydrous conditions to improve the reaction safety, so acetonitrile, tetrahydrofuran, acetone, pyridine, methyl ethyl ketone, N,N′-dimethylformamide, 1,4-dioxane, dimethyl sulfoxide and the like can be selected as the reaction solvent.

In some more preferred embodiments of the present disclosure, in step S1, the reaction is terminated by adding an organic phase and an aqueous phase to the reaction system; further preferably, a volume-to-mass ratio of the organic phase to paeoniflorin is 5:1 mL/g to 15:1 mL/g, and a volume-to-mass ratio of the aqueous phase to paeoniflorin is 2.5:1 mL/g to 7.5:1 mL/g; and further preferably, the organic phase is an ethyl acetate phase.

In some more preferred embodiments of the present disclosure, step S1 further comprises a step of extracting the reaction product with ethyl acetate and water after the reaction is terminated, and then performing concentration to obtain the crude product of paeoniflorin-6′-O-benzene sulfonate. After the termination of the reaction in step S1, by-products, organic salts, acid-binding agent and incompletely reacted substrates generally exist. Performing an extraction with ethyl acetate and water to obtain an extraction liquid can effectively remove some by-products and other impurities, thus obtaining a crude product of paeoniflorin-6′-O-benzene sulfonate with a higher content.

In some more preferred embodiments of the present disclosure, a filler in the column chromatography in S2 is silica gel, and a mass ratio of the silica gel to the crude product of paeoniflorin-6′-O)-benzene sulfonate is 4.5:1 to 9:1; and further preferably, the silica gel has a 100 to 300 mesh size; and further preferably, the silica gel has a 60 A to 90 A pore size.

In some more preferred embodiments of the present disclosure, the eluent for the column chromatography in step S2 is a mixed solvent of halogenated hydrocarbon and alcohol; further preferably, a volume ratio of the halogenated hydrocarbon to the alcohol is 8:1 to 15:1; and further preferably, the halogenated hydrocarbon is chloroform, dichloromethane or a combination thereof, and the alcohol is methanol, ethanol or a combination thereof.

Those skilled in the art know that in the case of conducting column chromatography for separation, the appearance of the main component indicates a starting point for collecting the eluate. while the appearance of impurities and the smaller spots of the main component indicate an end point for collecting the eluate.

In some more preferred embodiments of the present disclosure, a volume-to-mass ratio of the eluent to the crude product of paeoniflorin-6′-O-benzene sulfonate in step S2 is 45:1 mL/g to 65:1 mL/g.

In some more preferred embodiments of the present disclosure, in step S2, before the column chromatography, the crude product of paeoniflorin-6′-O-benzene sulfonate is dissolved in the eluent, and a volume-to-mass ratio of the eluent to the crude product of paeoniflorin-6′-O-benzene sulfonate is 1:1 mL/g to 3:1 mL/g.

In some more preferred embodiments of the present disclosure, the recrystallizing in a mixed solvent in step S3 comprises dissolving paeoniflorin-6′-O-benzene sulfonate obtained in step S2 in a good solvent, and then adding a poor solvent to a resulting solution crystallization.

In some more preferred embodiments of the present disclosure, a volume-to-mass ratio of the good solvent to paeoniflorin-6′-O)-benzene sulfonate obtained in step S2 is 8:1 mL/g to 16:1 mL/g; and further preferably, the good solvent is isopropyl acetate, isobutyl acetate or a combination thereof.

In some more preferred embodiments of the present disclosure, a volume-to-mass ratio of the poor solvent to paeoniflorin-6′-O-benzene sulfonate obtained in step S2 is 5:1 to 8:1 mL/g; and further preferably, the poor solvent is normal hexane, petroleum ether, iso-propyl ether, iso-octane or a combination thereof.

In some more preferred embodiments of the present disclosure, step S3 further comprises a step of filtering and drying a crystallized product.

The present disclosure has the following beneficial effects:
1. In the present disclosure, the crude product of paeoniflorin comprising paeoniflorin with a content of more than 40% is used as an initial raw material for synthesizing paeoniflorin-6′-O-benzene sulfonate, which greatly reduces the requirement on the content of paeoniflorin; and the preparation process is simple. The extract comprising paeoniflorin with a content of 40% has a low price (less than 0.1% of the price of paeoniflorin with a content of 98%), which can effectively reduce the industrial production cost while realizing the large-scale production.
2. In the present disclosure, the organometallic catalyst, the acid binding agent and the benzenesulfonyl chloride with active chemical properties are reacted with paeoniflorin. If paeoniflorin with a content of no less than 98% is used as the raw material, the yield can reach 75% or more. Compared with the prior art, less impurities are generated, less reaction solvent is needed. and the yield of paeoniflorin-6′-O-benzene sulfonate is significantly improved in the present disclosure. Both paeoniflorin with a content of 98% and paeoniflorin with a content of more than 40% are suitable as the raw material in this method, they may be subjected to column chromatography separation, purification and refining; then paeoniflorin-6′-O-benzene sulfonate with a content of 98% or more can be obtained.
3. In the present disclosure, during the purification of the crude product of paeoniflorin, an ethyl acetate-methanol or ethyl acetate-ethanol system is preferably used for the column chromatography separation of the crude product of paeoniflorin, which, compared with a halogenated hydrocarbon-alcohol system, requires less eluent and can improve the safety of industrial production.

### BRIEF DESCRIPTION OF THE DRAWINGS

The present disclosure will be further explained with reference to the drawings and embodiments hereinafter, wherein:

FIG. 1 shows an HPLC detection result after reaction in Comparative Example 1.

FIG. 2 shows HPLC detection results of the crude products of paeoniflorin-6′-O-benzene sulfonate in step S1 of Example 4 and Comparative Example 2.

### DETAILED DESCRIPTION

The concepts, technical features and technical effects of the present disclosure are clearly and completely described below in conjunction with the examples, such as to allow the objectives, features and effects of the present disclosure to be fully understood. Obviously, the described examples are merely some rather than all of the examples of the present disclosure. All other examples obtained by those skilled in the art based on the examples of the present disclosure without creative efforts shall fall within the protection scope of the present disclosure.

### Example 1

In this example, paeoniflorin-6′-O-benzene sulfonate was prepared, and the specific process was as follows:

Paeoniflorin purification: 50 g of crude product of paeoniflorin 1 (a water extract and alcohol precipitate of peony with a content of paeoniflorin of 44.7%) was taken and dissolved with 150 mL of methanol, then 100 g of neutral aluminium oxide was added for adsorption under stirring. The adsorbed neutral aluminium oxide was added in the chromatographic column, subjected to elution with 200 mL of methanol; and then the eluate was collected and concentrated to obtain 30.08 g of crude product of paeoniflorin II with a content of paeoniflorin of 74.1%. The obtained crude product of paeoniflorin II was dissolved in 100 ml of solvent (ethyl acetate:isopropyl alcohol was 3:1), and subjected to silica gel column chromatography, wherein 450 g of silica gel was loaded in the chromatographic column, the eluent for the column chromatography comprised ethyl acetate and isopropyl alcohol in a ratio of 3:1, and 1.75 L of eluent was used. The eluate was collected and concentrated under reduced pressure to obtain 24.1 g of paeoniflorin with a content of 85.3%.

S1: The obtained paeoniflorin was fully dissolved with 240 mL of acetone, added with 9.2 g of dimethyl tin dichloride and 8.5 mL of triethylamine, then further added with 6.5 g of benzenesulfonyl chloride, and a resulting mixture was stirred for reaction. After 2 hours of reaction, 240 mL of ethyl acetate and 150 mL of pure water were added and stirred for 5 minutes to terminate the reaction. After standing for 0.5 hour, an organic layer was collected and concentrated under reduced pressure to obtain 28.7 g of crude product of paeoniflorin-6′-O-benzene sulfonate with a content of 86.9%.

S2: The crude product of paeoniflorin-6′-O-benzene sulfonate was dissolved with 50 mL of solvent (chloroform:methanol was 10:1), subjected to silica gel column chromatography (monitored by TLC), wherein 175 g of silica gel was loaded in the chromatographic column, the eluent for the column chromatography comprised chloroform and methanol in a ratio of 10:1, and 1.30 L of eluent was used. A fraction containing only paeoniflorin-6′-O-benzene sulfonate was collected and concentrated under reduced pressure to obtain 21.5 g of paeoniflorin-6′-O-benzene sulfonate with a content of 97.9%.

S3: The above-mentioned paeoniflorin-6′-O-benzene sulfonate was completely dissolved with 200 mL of isopropyl acetate, added with 120 mL of normal hexane; then stirring, crystallization, filtration, rinse and drying were performed in sequence to obtain 19 g of paeoniflorin-6′-O-benzene sulfonate with a content of 99.4%.

### Example 2

In this example, paeoniflorin-6′-O-benzene sulfonate was prepared, and the specific process was as follows:

Paeoniflorin purification: 50 g of crude product of paeoniflorin 1 (a water extract and alcohol precipitate of peony with a content of paeoniflorin of 44.7%) was taken and dissolved with 50 mL of anhydrous ethanol, then 150 g of neutral aluminium oxide was added for adsorption under stirring. The adsorbed neutral aluminium oxide was added in the chromatographic column, subjected to elution with 300 mL of anhydrous ethanol, and then the eluate was collected and concentrated to obtain 29.4 g of crude product of paeoniflorin II with a content of paeoniflorin of 75.8%. The obtained crude product of paeoniflorin II was dissolved in 100 mL of solvent (ethyl acetate:ethanol was 4:1). and subjected to silica gel column chromatography; wherein 450 g of silica gel was loaded in the chromatographic column, the eluent for column chromatography comprised ethyl acetate and ethanol in a ratio of 4:1, and 1.75 L of eluent was used. The eluate was collected and concentrated under reduced pressure to obtain 23.5 g of paeoniflorin with a content of 84.1%.

S1: The obtained paeoniflorin was fully dissolved with 140 mL of acetonitrile, added with 0.4 g of diethyl tin dichloride and 12 g of potassium carbonate, then further added with 5.2 g of benzenesulfonyl chloride, and a resulting mixture was stirred for reaction. After 2 hours of reaction, 330 mL of ethyl acetate and 164 mL of pure water were added and stirred for 5 minutes to terminate the reaction. After standing for 0.5 hour, an organic layer was collected and concentrated under reduced pressure to obtain 25.2 g of crude product of paeoniflorin-6′-O-benzene sulfonate with a content of 78.6%.

S2: The crude product of paeoniflorin-6′-O-benzene sulfonate was dissolved with 50 mL of solvent (chloroform:ethanol was 12:1), and subjected to silica gel column chromatography (monitored by TLC), wherein 175 g of silica gel was loaded in the chromatographic column, the eluent for column chromatography comprised chloroform and methanol in a ratio of 12:1, and 1.30 L of eluent was used. A fraction containing only paeoniflorin-6′-O-benzene sulfonate was collected and concentrated under reduced pressure to obtain 16.63 g of paeoniflorin-6′-O-benzene sulfonate with a content of 96.8%.

S3: The above-mentioned paeoniflorin-6′-O-benzene sulfonate was completely dissolved with 240 mL of isopropyl acetate, added with 120 mL of iso-octane, then stirring, crystallization, filtration, rinse and drying were performed in sequence to obtain 15.7 g of paeoniflorin-6′-O-benzene sulfonate with a content of 98.8%.

### Example 3

In this example, paeoniflorin-6′-O-benzene sulfonate was prepared, and the specific process was as follows:

Paeoniflorin purification: 50 g of crude product of paeoniflorin 1 (a water extract and alcohol precipitate of peony with a content of paeoniflorin of 44.7%) was taken and dissolved with 100 mL of isopropyl alcohol, then 150 g of neutral aluminium oxide was added for adsorption under stirring. The adsorbed neutral aluminium oxide was added in the chromatographic column, subjected to elution with 200 mL of isopropyl alcohol, and then the eluate was collected and concentrated to obtain 28.4 g of crude product of paeoniflorin II with a content of paeoniflorin of 78.3%. The obtained crude product of paeoniflorin II was dissolved in 100 mL of solvent (chloroform:ethanol was 5:1) and subjected to silica gel column chromatography, wherein 450 g of silica gel was loaded in the chromatographic column, the eluent for column chromatography comprised chloroform and ethanol in a ratio of 9:1, and 2.75 L of eluent was used. The eluate was collected and concentrated under reduced pressure to obtain 23.7 g of paeoniflorin with a content of 90.5%.

S1: The obtained paeoniflorin was fully dissolved with 80 mL of pyridine, added with 0.18 g of diphenyl tin dichloride and 6.5 mL of N,N′-diisopropylethylamine, then further added with 13 g of benzenesulfonyl chloride, and a resulting mixture was stirred for reaction. After 2 hours of reaction, 140 mL of ethyl acetate and 70 mL of pure water were added and stirred for 5 minutes to terminate the reaction. After standing for 0.5 hour, an organic layer was collected and concentrated under reduced pressure to obtain 26.9 g of crude product of paeoniflorin-6′-O-benzene sulfonate with a content of 75.9%.

S2: The crude product of paeoniflorin-6′-O-benzene sulfonate was dissolved with 50 mL of solvent (chloroform:methanol was 12:1), and subjected to silica gel column chromatography (monitored by TLC), wherein 240 g of silica gel was loaded in the chromatographic column, the eluent for column chromatography comprised chloroform and methanol in a ratio of 14:1, and 1.60 L of eluent was used. A fraction containing only paeoniflorin-6′-O-benzene sulfonate was collected and concentrated under reduced pressure to obtain 16.21 g of paeoniflorin-6′-O-benzene sulfonate with a content of 95.8%.

S3: The above-mentioned paeoniflorin-6′-O-benzene sulfonate was completely dissolved with 150 ml of isopropyl acetate, added with 900 mL of iso-propyl ether, then stirring, crystallization, filtration, rinse and drying were performed in sequence to obtain 15.3 g of paeoniflorin-6′-O)-benzene sulfonate with a content of 98.2%.

### Example 4

In this example, paeoniflorin-6′-O-benzene sulfonate was prepared, and the specific process was as follows:

Paeoniflorin purification: 120 g of crude product of paeoniflorin 1 (a water extract and alcohol precipitate of peony with a content of paeoniflorin of 44.7%) was taken and dissolved with 240 mL of isopropyl alcohol, then subjected to aluminium oxide column chromatography with 600 mL of isopropyl alcohol; wherein 360 g of neutral aluminium oxide was loaded in the chromatographic column. Then the eluate was collected and concentrated to obtain 70.1 g of crude product of paeoniflorin II with a content of paeoniflorin of 76.3%. The obtained crude product of paeoniflorin II was dissolved in 240 mL of solvent (ethyl acetate:methanol was 3:1), and subjected to silica gel column chromatography, wherein 1,440 g of silica gel was loaded in the chromatographic column, the eluent for column chromatography comprised ethyl acetate and methanol in a ratio of 3:1, and 4.2 L of eluent was used. The eluate was collected and concentrated under reduced pressure to obtain 58.8 g of paeoniflorin with a content of 85.1%.

S1: The obtained paeoniflorin was fully dissolved with 180 mL of methyl ethyl ketone, added with 0.4 g of dimethyl tin oxide and 12 mL of pyridine, then further added with 29.4 g of benzenesulfonyl chloride, and a resulting mixture was stirred for reaction. After the reaction, 480 mL of ethyl acetate and 240 mL of pure water were added and stirred for $ minutes to terminate the reaction. After standing for 0.75 hour, an organic layer was collected and concentrated under reduced pressure to obtain 74.0 g of crude product of paeoniflorin-6′-O-benzene sulfonate with a content of 76.7%.

S2: The crude product of paeoniflorin-6′-O-benzene sulfonate was dissolved with 120 ml of solvent (chloroform:ethanol=14:1), subjected to silica gel column chromatography (monitored by TLC), wherein 520 g of silica gel was loaded in the chromatographic column, the eluent for column chromatography comprised chloroform and ethanol in a ratio of 14:1, and 3.7 L of eluent was used. A fraction containing only paeoniflorin-6′-O-benzene sulfonate was collected and concentrated under reduced pressure to obtain 45.4 g of paeoniflorin-6′-O-benzene sulfonate with a content of 95.9%.

S3: The above-mentioned paeoniflorin-6′-O-benzene sulfonate was completely dissolved with 600 mL of isopropyl acetate, added with 310 mL of normal hexane, then stirring, crystallization, filtration, rinse and drying were performed in sequence to obtain 42.8 g of paeoniflorin-6′-O-benzene sulfonate with a content of 98.5%.

### Example 5

In this example, paeoniflorin-6′-O-benzene sulfonate was prepared, and the specific process was as follows:

Paeoniflorin purification: 500 g of crude product of paeoniflorin 1 (a water extract and alcohol precipitate of peony with a content of paeoniflorin of 44.7%) was taken and dissolved with 1.5 L of methanol, then subjected to aluminium oxide column chromatography with 1.5 L of methanol, wherein 1 kg of neutral aluminium oxide was loaded in the chromatographic column. Then the eluate was collected and concentrated to obtain 296 g of crude product of paeoniflorin II with a content of 75.1%. The obtained crude product of paeoniflorin II was dissolved in 1 L of solvent (chloroform: ethanol was 5:1) and subjected to silica gel column chromatography; wherein 2.5 kg of silica gel was loaded in the chromatographic column, the eluent for column chromatography comprised ethyl acetate and ethanol in a ratio of 14:1, and 27.5 L of eluent was used. The eluate was collected and concentrated under reduced pressure to obtain 240 g of paeoniflorin with a content of 87.2%.

S1: The obtained paeoniflorin was fully dissolved with 0.75 L of tetrahydrofuran, added with 1.5 g of di-tert-butyl tin dichloride and 90 mL of N,N′-diisopropylethylamine, then further added with 96 g of benzenesulfonyl chloride, and a resulting mixture was stirred for reaction. After the reaction, 1.5 L of ethyl acetate and 1 L of pure water were added and stirred for 5 minutes to terminate the reaction. After standing for 1 hour, an organic layer was collected and concentrated under reduced pressure to obtain 289 g of crude product of paeoniflorin-6′-O-benzene sulfonate with a content of 77.5%.

S2: The crude product of paeoniflorin-6′-O-benzene sulfonate was dissolved with 500 mL of solvent (chloroform:ethanol was 13:1), subjected to silica gel column chromatography (monitored by TLC), wherein 1.5 kg of silica gel was loaded in the chromatographic column, the eluent for column chromatography comprised chloroform and ethanol in a ratio of 13:1, and 18.9 L of eluent was used. A fraction containing only paeoniflorin-6′-O-benzene sulfonate was collected and concentrated under reduced pressure to obtain 183 g of paeoniflorin-6′-O-benzene sulfonate with a content of 96.1%.

S3: The above-mentioned paeoniflorin-6′-O-benzene sulfonate was completely dissolved with 1.8 L of isopropyl acetate, added with 1 L of iso-octane, then stirring, crystallization, filtration, rinse and drying were performed in sequence to obtain 172.6 g of paeoniflorin-6′-O-benzene sulfonate with a content of 98.8%.

### Example 6

In this example, paeoniflorin-6′-O)-benzene sulfonate was prepared, and the specific process was as follows:

Paeoniflorin purification: 1 kg of crude product of paeoniflorin 1 (a water extract and alcohol precipitate of peony with a content of paeoniflorin of 44.7%) was taken and dissolved with 2 L of anhydrous ethanol, then 3 kg of neutral aluminium oxide was added for adsorption under stirring. The adsorbed neutral aluminium oxide was added in the chromatographic column, subjected to elution with 6 L of anhydrous ethanol, and then the eluate was collected and concentrated to obtain 573 g of crude product of paeoniflorin II with a content of 77.6%. The obtained crude product of paeoniflorin was dissolved in 2 L of solvent (ethyl acetate:isopropyl alcohol was 4:1), and subjected to silica gel column chromatography, wherein 6 kg of silica gel was loaded in the chromatographic column, the eluent for the column chromatography comprised ethyl acetate and isopropyl alcohol in a ratio of 4:1, and 35 L of eluent was used. The eluate was collected and concentrated under reduced pressure to obtain 485 g of paeoniflorin with a content of 82.6%.

S1: The obtained paeoniflorin was fully dissolved with 1.2 L of dimethyl sulfoxide, added with 4 g of dimethyl tin sulfide and 110 g of sodium carbonate, then further added with 107 g of benzenesulfonyl chloride, and a resulting mixture was stirred for reaction. After the reaction. 3.5 L of ethyl acetate and 2 L of pure water were added and stirred for 5 minutes to terminate the reaction. After standing for 1.5 hours, an organic layer was collected and concentrated under reduced pressure to obtain 531 g of crude product of paeoniflorin-6′-O)-benzene sulfonate with a content of 79.6%.

S2: The crude product of paeoniflorin-6′-O-benzene sulfonate was dissolved with 1 L of solvent (chloroform:ethanol was 10:1), subjected to silica gel column chromatography (monitored by TLC), wherein 4.7 kg of silica gel was loaded in the chromatographic column, the eluent for column chromatography comprised chloroform and ethanol in a ratio of 10:1, and 24 L of eluent was used. A fraction containing only paeoniflorin-6′-O-benzene sulfonate was collected and concentrated under reduced pressure to obtain 342 g of paeoniflorin-6′-O-benzene sulfonate with a content of 97.1%.

S3: The above-mentioned paeoniflorin-6′-O-benzene sulfonate was completely dissolved with 4 L of isopropyl acetate, added with 1.8 L of iso-octane, then stirring, crystallization, filtration, rinse and drying were performed in sequence to obtain 322.5 g of paeoniflorin-6′-O-benzene sulfonate with a content of 98.3%.

### Example 7

In this example, paeoniflorin-6′-O-benzene sulfonate was prepared, and the specific process was as follows:

Paeoniflorin purification: 6 kg of crude product of paeoniflorin 1 (a water extract and alcohol precipitate of peony with a content of paeoniflorin of 44.7%) were taken and dissolved with 6 L of methanol, then 12 kg of neutral aluminium oxide was added for adsorption under stirring. The adsorbed neutral aluminium oxide was added in the chromatographic column, subjected to elution with 24 L of methanol, then the eluate was collected and concentrated to obtain 3.28 kg of crude product of paeoniflorin II with a content of 76.0%. The obtained crude product of paeoniflorin was dissolved in 12 L of solvent (ethyl acetate:methanol was 4:1), and subjected to silica gel column chromatography, wherein 30 kg of silica gel was loaded in the chromatographic column, the eluent for the column chromatography comprised ethyl acetate and methanol in a ratio of 6:1, and 210 L of eluent was used. The eluate was collected and concentrated under reduced pressure to obtain 2.76 kg of paeoniflorin with a content of 81.8%.

S1: The obtained paeoniflorin was fully dissolved with 7.2 L of acetonitrile, added with 18 g of dimethyl tin dichloride and 796 ml of triethylamine, then further added with 770 g of benzenesulfonyl chloride, and a resulting mixture was stirred for reaction. After the reaction, 24 L of ethyl acetate and 12 L of pure water were added and stirred for 5 minutes to terminate the reaction. After standing for 1.5 hours, an organic layer was collected and concentrated under reduced pressure to obtain 3.14 kg of crude product of paeoniflorin-6-O-benzene sulfonate with a content of 82.1%.

S2: The crude product of paeoniflorin-6′-O-benzene sulfonate was dissolved with 1 L of solvent (chloroform:ethanol was 12:1), and subjected to silica gel column chromatography (monitored by TLC), wherein 21 kg of silica gel was loaded in the chromatographic column, the eluent for the column chromatography comprised chloroform and ethanol in a ratio of 12:1, and 145 L of eluent was used. A fraction containing only paeoniflorin-6′-O-benzene sulfonate was collected and concentrated under reduced pressure to obtain 2.21 kg of paeoniflorin-6′ -O-benzene sulfonate with a content of 97.6%.

S3: The above-mentioned paeoniflorin-6 -O-benzene sulfonate was completely dissolved with 30 L of isobutyl acetate, added with 13 L of normal hexane, then stirring, crystallization, filtration, rinse and drying were performed in sequence to obtain 2.08 kg of paeoniflorin-6′-O-benzene sulfonate with a content of 99.3%.

### Example 8

In this example, paeoniflorin-6′-O-benzene sulfonate was prepared, and the specific process was as follows:

Paeoniflorin purification: 1 kg of crude product of paeoniflorin 1 (a water extract and alcohol precipitate of peony with a content of paeoniflorin of 55.8%) were taken and dissolved with 2 L of anhydrous ethanol, then 2 kg of neutral aluminium oxide was added for adsorption under stirring. The adsorbed neutral aluminium oxide was added in the chromatographic column, subjected to elution with 5 L of anhydrous ethanol, then the eluate was collected and concentrated to obtain 695 g of crude product of paeoniflorin II with a content of 74.6%. The obtained crude product of paeoniflorin was dissolved in 2 L of solvent (ethyl acetate:isopropyl alcohol=4:1), and subjected to silica gel column chromatography, wherein 6 kg of silica gel was loaded in the chromatographic column, the eluent for the column chromatography comprised ethyl acetate and isopropyl alcohol in a ratio of 6:1, and 35 L of eluent was used. The eluate was collected and concentrated under reduced pressure to obtain 471 g of paeoniflorin with a content of 93.6%.

S1: The obtained paeoniflorin was fully dissolved with 1.2 L of acetone, added with 4 g of dimethyl tin dichloride and 110 g of potassium carbonate, then further added with 107 g of benzenesulfonyl chloride, and a resulting mixture was stirred for reaction. After the reaction, 3.5 L of ethyl acetate and 2 L of pure water were added and stirred for 5 minutes to terminate the reaction. After standing for 1.5 hours, an organic layer was collected and concentrated under reduced pressure to obtain 547 g of crude product of paeoniflorin-6′-O-benzene sulfonate with a content of 88.7%.

S2: The crude product of paeoniflorin-6′-O)-benzene sulfonate was dissolved with 1 L of solvent (chloroform:ethanol was 4:1), and subjected to silica gel column chromatography (monitored by TLC), wherein 4.8 kg of silica gel was loaded in the chromatographic column, the eluent for the column chromatography comprised chloroform and ethanol in a ratio of 12:1, and 24 L of eluent was used. A fraction containing only paeoniflorin-6′-O-benzene sulfonate was collected and concentrated under reduced pressure to obtain 459 g of paeoniflorin-6′-O-benzene sulfonate with a content of 97.8%.

S3: The above-mentioned paeoniflorin-6′-O-benzene sulfonate was completely dissolved with 4 L of isobutyl acetate, added with 1.8 L of normal hexane, then stirring, crystallization, filtration, rinse and drying were performed in sequence to obtain 417 g of paeoniflorin-6′-O-benzene sulfonate with a content of 99.3%.

### Example 9

In this example, paeoniflorin-6′-O-benzene sulfonate was prepared, and the specific process was as follows:

Paeoniflorin purification: 1 kg of crude product of paeoniflorin 1 (a water extract and alcohol precipitate of peony with a content of paeoniflorin of 67.1%) were taken and dissolved with 2 L of anhydrous ethanol, then 3 kg of neutral aluminium oxide was added for adsorption under stirring. The adsorbed neutral aluminium oxide was added in the chromatographic column, subjected to elution with 5 L of anhydrous ethanol, and then the eluate was collected and concentrated to obtain 815 g of crude product of paeoniflorin II with a content of 81.2%. The obtained crude product of paeoniflorin was dissolved in 2 L of solvent (ethyl acetate:ethanol was 3:1), and subjected to silica gel column chromatography, wherein 5.6 kg of silica gel was loaded in the chromatographic column, the eluent for the column chromatography comprised ethyl acetate and ethanol in a ratio of 5:1, and 35 L of eluent was used. The eluate was collected and concentrated under reduced pressure to obtain 555 g of paeoniflorin with a content of 95.2%.

S1: The obtained paeoniflorin was fully dissolved with 1.2 L of acetonitrile, added with 4.4 g of dimethyl tin oxide and 125 mL of triethylamine, then further added with 111 g of benzenesulfonyl chloride, and a resulting mixture was stirred for reaction. After the reaction, 5.5 L of ethyl acetate and 2.5 L of pure water were added and stirred for 5 minutes to terminate the reaction. After standing for 1.5 hours, an organic layer was collected and concentrated under reduced pressure to obtain 630 g of crude product of paeoniflorin-6′-O-benzene sulfonate with a content of 90.1%.

S2: The crude product of paeoniflorin-6′-O-benzene sulfonate was dissolved with 1 L of solvent (chloroform:methanol=12:1), and subjected to silica gel column chromatography (monitored by TLC), wherein 4.8 kg of silica gel was loaded in the chromatographic column, the eluent for the column chromatography comprised chloroform and methanol in a ratio of 12:1, and 25 L of eluent was used. A fraction containing only paeoniflorin-6′-O-benzene sulfonate was collected and concentrated under reduced pressure to obtain 550 g of paeoniflorin-6′-O-benzene sulfonate with a content of 98%.

S3: The above-mentioned paeoniflorin-6′-O-benzene sulfonate was completely dissolved with 4.4 L of isobutyl acetate, added with 4.4 L of normal hexane, then stirring, crystallization, filtration, rinse and drying were performed in sequence to obtain 523 g of paeoniflorin-6′-O)-benzene sulfonate with a content of 99.4%.

### Example 10

In this example, paeoniflorin-6′-O-benzene sulfonate was prepared, and the specific process was as follows:

Paeoniflorin purification: 60 kg of crude product of paeoniflorin 1 (a water extract and alcohol precipitate of peony with a content of paeoniflorin of 44.7%) were taken and dissolved with 120 L of anhydrous ethanol, then 180 kg of neutral aluminium oxide was added for adsorption under stirring. The adsorbed neutral aluminium oxide was added in the chromatographic column, subjected to elution with 240 L of anhydrous ethanol, and then the eluate was collected and concentrated to obtain 36.1 kg of crude product of paeoniflorin II with a content of 77.0%. The obtained crude product of paeoniflorin was dissolved in 120 L of solvent (ethyl acetate:ethanol was 5:1), and subjected to silica gel column chromatography, wherein 480 kg of silica gel was loaded in the chromatographic column, the eluent for the column chromatography comprised ethyl acetate and ethanol in a ratio of 5:1, and 2,400 L of eluent was used. The eluate was collected and concentrated under reduced pressure to obtain 28.9 kg of paeoniflorin with a content of 88.8%.

S1: The obtained paeoniflorin was fully dissolved with 120 L of acetone, added with 235 g of dimethyl tin dichloride and 12 L of N,N′-diisopropylethylamine, then further added with 8.6 kg of benzenesulfonyl chloride, and a resulting mixture was stirred for reaction. After the reaction, 294 L of ethyl acetate and 147 L of pure water were added and stirred for 5 minutes to terminate the reaction. After standing for 2 hours, an organic layer was collected and concentrated under reduced pressure to obtain 37.5 kg of crude product of paeoniflorin-6′-O-benzene sulfonate with a content of 81.3%.

S2: The crude product of paeoniflorin-6′-O-benzene sulfonate was dissolved with 100 ml of solvent (chloroform:ethanol was 13:1), and subjected to silica gel column chromatography (monitored by TLC), wherein 300 kg of silica gel was loaded in the chromatographic column, the eluent for the column chromatography comprised chloroform and ethanol in a ratio of 13:1, and 2,000 L of eluent was used. A fraction containing only paeoniflorin-6′-O-benzene sulfonate was collected and concentrated under reduced pressure to obtain 24.5 kg of paeoniflorin-6′-O-benzene sulfonate with a content of 97.4%.

S3: The above-mentioned paeoniflorin-6′-O-benzene sulfonate was completely dissolved with 240 L of isobutyl acetate, added with 145 L of normal hexane, then stirring, crystallization, filtration, rinse and drying were performed in sequence to obtain 23.1 kg of paeoniflorin-6′-O-benzene sulfonate with a content of 99.1%.

### Example 11

In this example, paeoniflorin-6′-O)-benzene sulfonate was prepared, and the specific process was as follows:

Paeoniflorin purification: 120 kg of crude product of paeoniflorin 1 (a water extract and alcohol precipitate of peony with a content of paeoniflorin of 44.7%) were taken and dissolved with 120 L of anhydrous ethanol, then 240 kg of neutral aluminium oxide was added for adsorption under stirring. The adsorbed neutral aluminium oxide was added in the chromatographic column, subjected to elution with 600 L of anhydrous ethanol, and then the eluate was collected and concentrated to obtain 75.4 kg of crude product of paeoniflorin II with a content of 74.6%. The obtained crude product of paeoniflorin was dissolved in 240 L of solvent (ethyl acetate:methanol was 5:1), and subjected to silica gel column chromatography, wherein 840 kg of silica gel was loaded in the chromatographic column, the eluent for the column chromatography comprised ethyl acetate and methanol in a ratio of 5:1, and 4,200 L of eluent was used. The eluate was collected and concentrated under reduced pressure to obtain 59.2 kg of paeoniflorin with a content of 87.2%.

S1: The obtained paeoniflorin was dissolved with 240 L of acetone, added with 590 g of dimethyl tin dichloride and 27 L of triethylamine, then further added with 16.6 kg of benzenesulfonyl chloride, and a resulting mixture was stirred for reaction. After the reaction, 480 L of ethyl acetate and 240 L of pure water were added and stirred for 5 minutes to terminate the reaction. After standing for 2 hours, an organic layer was collected and concentrated under reduced pressure to obtain 72.2 kg of crude product of paeoniflorin-6′-O-benzene sulfonate with a content of 80.4%.

S2: The crude product of paeoniflorin-6′-O-benzene sulfonate was dissolved with 200 L of solvent (chloroform:ethanol was 12:1), and subjected to silica gel column chromatography (monitored by TLC), wherein 400 kg of silica gel was loaded in the chromatographic column, the eluent for the column chromatography comprised chloroform and ethanol in a ratio of 12:1, and 3,500 L of eluent was used. A fraction containing only paeoniflorin-6′-O-benzene sulfonate was collected and concentrated under reduced pressure to obtain 46.4 kg of paeoniflorin-6′-O-benzene sulfonate with a content of 97.2%.

S3: The above-mentioned paeoniflorin-6 -O-benzene sulfonate was completely dissolved with 500 L of isobutyl acetate, added with 360 L of normal hexane, then stirring, crystallization, filtration, rinse and drying were performed in sequence to obtain 43.76 kg of paeoniflorin-6′-O-benzene sulfonate with a content of 98.9%.

From the above examples, it could be found that the technical solution provided by the present disclosure could be used in industrial production, and the reaction solvent and eluent were used in an amount within the scope of the claims, and had good stability.

From Example 1 to Example 3. it could be seen that in the purification step of paeoniflorin, the chloroform-methanol system was conducive to a good separation effect, but a high-fold amount of solvent used was required, so the ethyl acetate-alcohol system was more suitable for mass preparation.

From Example 1 to Example 7, it could be seen that the reaction solvent and eluent for gradually amplifying the reaction were used in amounts within the scope of the claims, and had good stability.

From Example 7 to Example 9, it could be seen that the higher the initial contents of raw materials, the higher the yield and content of paeoniflorin-6′-O-benzene sulfonate prepared by the present disclosure.

From Example 10 and Example 11, it could be seen that the reaction solvent and eluent for industrial production were used in amounts within the scope of the claims, and had good stability.

### Comparative Example 1

In this comparative example, paeoniflorin with a content of paeoniflorin of 98% instead of the crude product of paeoniflorin was used as an initial material to prepare paeoniflorin-6′-O-benzene sulfonate, and the specific process was as follows:

### Prior Art:

S1: 6 g of paeoniflorin with a content of paeoniflorin of 98% was taken and added into a reaction bottle, then added with a mixed solution consisting of 36 mL of chloroform and 8 mL of pyridine for dissolution, and further added with 50 mg of 4-dimethylaminopyridine for activation for 30 minutes. Within 3 hours, 14.4 g of benzenesulfonyl chloride was dissolved in 4 mL of dichloromethane, then a resulting mixture was dropped into the reaction solution for reaction at a reaction temperature of 35□. After dropping, the reaction continued for 8 hours, and the reaction process was monitored by TLC. After the reaction was completed, the reaction system was concentrated into an oily matter, then extracted thrice with 60 mL of ethyl acetate, and each time added with 60 mL of 1M hydrochloric acid. An organic layer was collected and concentrated to obtain 5.9 g of crude product of paeoniflorin-6′-O-benzene sulfonate, with a content of 76.8% and a yield of 59.6%.

S2: The crude product of paeoniflorin-6′-O-benzene sulfonate was dissolved with 12 mL of solvent (chloroform:absolute ethanol was 3:1), and separated by column chromatography filled with 36 g of silica gel (the eluent comprised chloroform and methanol in a ratio of 35:1) to obtain 3.1 g of paeoniflorin-6′-O-benzene sulfonate with a content of 98.3%, wherein 9.95 L of eluent was used.

### Process of the Present Disclosure:

S1: 5 g of paeoniflorin with a content of 98% was fully dissolved with 25 mL of acetone, added with 0.04 g of dimethyltin dichloride, the resulting solution was allowed to stand for 10 minutes, then further added with 2.5 mL of triethylamine and 2 g of benzenesulfonyl chloride in sequence; a resulting mixture was stirred for 2 h. The reaction progression was detected by TLC. After the reaction was completed, 60 mL of ethyl acetate and 30 mL of pure water were added and stirred for 15 minutes to terminate the reaction. After standing for 0.5 hour, an organic layer was collected and concentrated under reduced pressure to obtain 6.22 g of crude product of paeoniflorin-6′-O-benzene sulfonate with a content of 91.2%.

S2: The crude product of paeoniflorin-6′-O-benzene sulfonate was dissolved with 12 mL of solvent (chloroform:anhydrous ethanol was 3:1), and subjected to silica gel column chromatography (monitored by TLC), wherein 36 g of silica gel was loaded in the chromatographic column, the eluent comprised chloroform and ethanol in a ratio of 12:1, and 275 mL of eluent was used. A fraction containing only paeoniflorin-6′-O-benzene sulfonate was collected and concentrated under reduced pressure to obtain 5.22 g of paeoniflorin-6′-O-benzene sulfonate with a content of 98.3% and a yield of 79.4%.

S3: The above-mentioned paeoniflorin-6 -O-benzene sulfonate was completely dissolved with 32 mL of isopropyl acetate, added with 18 mL of iso-octane for crystallization under stirring, and then filtration, rinse and drying were performed in sequence to obtain 4.93 g of paeoniflorin-6′-O-benzene sulfonate with a content of 99.6%.

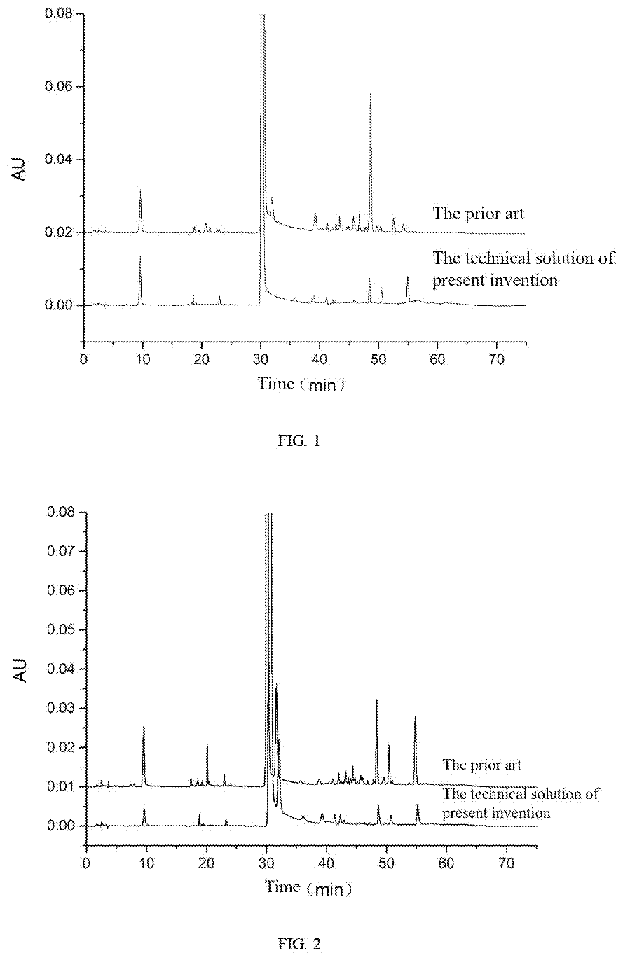

From Comparative Example 1, it could be found that paeoniflorin with a content higher than 98% was directly used as the raw material in the process of present disclosure, which, compared with the prior art, achieved a much higher yield (75% or more) and generated less impurities after the reaction (as shown in FIG. 1), and was easier to prepare paeoniflorin-6′-O)-benzene sulfonate with a high-content by silica gel column chromatography. The eluent for the column chromatography comprising chloroform and methanol in a ratio of 10:1 to 14:1 possessed a stronger polarity and elution ability compared with the eluent comprising chloroform and methanol in a ratio of 20:1 to 45:1 used in the prior art, so that paeoniflorin-6′-O)-benzene sulfonate with a high content could be obtained, less solvent was used, and the separation efficiency could be improved.

### Comparative Example 2

In this comparative example, a crude product of paeoniflorin with a content of paeoniflorin of 44.7% was used as an initial material. The crude product of paeoniflorin was separated first to obtain self-prepared paeoniflorin, and paeoniflorin-6′-O-benzene sulfonate was prepared in the presence of 4-dimethylaminopyridine (DMAP) as a catalyzer. The specific process was as follows:

120 g of crude product of paeoniflorin 1 were taken, dissolved with 240 mL of isopropyl alcohol, then subjected to the aluminium oxide chromatographic column, wherein 360 g of neutral aluminium oxide was loaded in the chromatographic column, and 600 mL of isopropyl alcohol was used. The eluate was collected and concentrated to obtain 70.1 g of crude product of paeoniflorin II with a content of paeoniflorin of 76.3%. The obtained crude product of paeoniflorin II was dissolved in 240 mL of solvent (ethyl acetate:methanol was 3:1), and subjected to silica gel column chromatography, wherein 1,440 g of silica gel was loaded in the chromatographic column, the eluent comprised ethyl acetate and methanol in a ratio of 3:1, and 4.2 L of eluent was used. The eluate was collected and concentrated under reduced pressure to obtain 58.8 g of paeoniflorin with a content of 85.1%.

S1: 6 g of obtained paeoniflorin was added into a reaction bottle, dissolved with a mixed solution consisting of 36 mL of chloroform and 8 mL of pyridine, and then added with 50 mg of 4-dimethylaminopyridine for activation for 30 minutes. Within 3 hours, 14.4 g of benzenesulfonyl chloride was dissolved in 4 mL of dichloromethane, then a resulting mixture was dropped into the reaction solution for reaction at a reaction temperature of 35° C. After dropping, the reaction continued for 8 hours, and the reaction progression was monitored by TLC. After the reaction was completed, the reaction system was concentrated into an oily matter, then extracted thrice with 60 mL of ethyl acetate, and each time added with 60 mL of 1M hydrochloric acid. An organic layer was collected and concentrated to obtain 6.2 g of crude product of paeoniflorin-6′-O-benzene sulfonate with a content of 50.6%.

S2: The crude product of paeoniflorin-6′-O-benzene sulfonate was dissolved with 12 mL of solvent (chloroform:absolute ethanol was 3:1), and separated by column chromatography, wherein 36 g of silica gel was loaded in the chromatographic column, the eluent comprised chloroform and methanol in a ratio of 35:1, and 9.95 L of eluent was used. Then, 3.33 g of paeoniflorin-6′-O-benzene sulfonate with a content of 94.3% was obtained.

From Comparative Examples 1 to 2, it could be seen that paeoniflorin with a content of higher than 98% was directly used as a raw material in the preparation process of paeoniflorin-6′-O-benzene sulfonate, which, compared with the prior art, achieved a higher yield and purity, generated less impurities, required less reaction solvent, and led to a more than 3-fold decrease in the amount of eluent for column chromatography. The paeoniflorin separated directly by the method of the present disclosure was subjected to a reaction according to the prior art, and the yield and content achieved were lower than that of the present disclosure (as shown in FIG. 2) regardless of the amount of the solvent.

The embodiments of the present disclosure are described in detail above, but the present disclosure is not limited to the above embodiments, and various changes may also be made within the knowledge scope of those of ordinary skills in the art without departing from the purpose of the present disclosure. In addition, in case of no conflict, the embodiments in the present disclosure and the features in the embodiments may be combined with each other.

1. A preparation method of paeoniflorin-6-O-benzene sulfonate of a following formula, 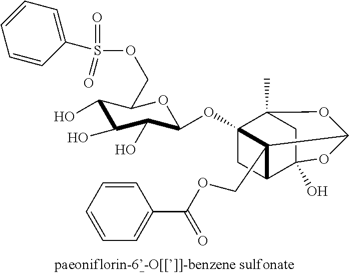
comprising the following steps of:
S1: adding an organic solvent, an organometallic catalyst, an acid binding agent and bensenesulfonyl chloride into paeoniflorin for reaction, and concentrating a reaction product after the reaction is terminated to obtain a crude product of paeoniflorin-6′-O-benzene sulfonate;
S2: separating the crude product of paeoniflorin-6-O-benzene sulfonate by column chromatography, and collecting an eluate to obtain paeoniflorin 6′-O-benzene sulfonate with a content of at least 95%;
S3: recrystallizing paeoniflorin 6′-O-benzene sulfonate obtained in step S2 in a mixed solvent to obtain paeoniflorin-6′-O-benzene sulfonate with a content of at least 98%.

2. The preparation method of paeoniflorin-6′-O-benzene sulfonate according to claim 1, wherein before step S1, the method further comprises a step of separating a crude product of paeoniflorin by column chromatography to obtain paeoniflorin with a content of at least 80%.

3. The preparation method of paeoniflorin-6′-O-benzene sulfonate according to claim 2, wherein an eluent for the column chromatography is a mixed solvent of alkane derivative and lower alcohol.

4. The preparation method of paeoniflorin-6′-O-benzene sulfonate according to claim 2, wherein the crude product of paeoniflorin is derived from a natural plant extract with a paeoniflorin content of greater than 40%.

5. The preparation method of paeoniflorin-6′-O-benzene sulfonate according to claim 3, wherein in the mixed solvent of alkane derivative and lower alcohol, a volume ratio of the alkane derivative to the lower alcohol is 3:1 to 15:1.

6. The preparation method of paeoniflorin-6′-O-benzene sulfonate according to claim 3, wherein in the column chromatography, a volume-to-mass ratio of the eluent to the crude product of paeoniflorin is 35:1 mL/g to 55:1 mL/g.

7. The preparation method of paeoniflorin-6′-O-benzene according to claim 3, wherein a filler in the column chromatography is silica gel.

8. The preparation method of paeoniflorin-6′-O-benzene sulfonate according to claim 2, wherein before the column chromatography, the method further comprises a step of carrying out dissolution, adsorption, elution and concentration on the crude product of paeoniflorin.

9. The preparation method of paeoniflorin-6′-O-benzene sulfonate according to claim 8, wherein the adsorption comprises adsorption under stirring and/or column chromatography adsorption.

10. The preparation method of paeoniflorin-6′-O-benzene sulfonate according to claim 1, wherein the organometallic catalyst in step S1 is at least one selected frons the group consisting of C₁-C₄ alkyl tin halide, aryl tin halide, C₁-C₄ alkyl tin oxide and C₁-C₄ alkyl tin sulfide.

11. The preparation method of paeoniflorin-6′-O-benzene sulfonate according to claim 1, wherein the organometallic catalyst in step S1 is added in an amount of 0.035% to 45% by weight of paeoniflorin.

12. The preparation method of paeoniflorin-6′-O-benzene sulfonate according to claim 1, wherein the organic solvent for the reaction in step S1 is at least owe selected from the group consisting of acetonitrile, tetrahydrofuran, acetone, pyridine, methyl ethyl ketone, N,N′-dimethylformamide, 1,4 dioxane and dimethyl sulfoxide.

13. The preparation method of paeoniflorin-6′-O-benzene sulfonate according to claim 1, wherein the acid binding agent in step S1 is triethylamine, N,N′-diisopropylethylamine, pyridine, potassium carbonate, potassium bicarbonate, sodium carbonate, sodium bicarbonate or a combination thereof.

14. The preparation method of paeoniflorin-6′-O-benzene sulfonate according to claim 1, wherein step S1 further comprises a step of extracting the reaction product with ethyl acetate and water after the reaction is terminated, and then performing concentration to obtain the crude product of paeoniflorin-6′-O-benzene sulfonate.

15. The preparation method of paeoniflorin-6′-O-benzene sulfonate according to claim 1, wherein the eluent for the column chromatography in step S2 is a mixed solvent of halogenated hydrocarbon and lower alcohol.

16. The preparation method of paeoniflorin-6′-O-benzene sulfonate according to claim 1, wherein the recrystallizing in a mixed solvent in step S3 comprises dissolving paeoniflorin-6′-O-benzene sulfonate obtained in step S3 in a good solvent, and then adding a poor solvent to a resulting solution for crystallization.

17. The preparation method of paeoniflorin-6′-O-benzene sulfonate according to claim 16, wherein the good solvent is isopropyl acetate, isobutyl acetate or a combination thereof.

18. The preparation method of paeoniflorin-6′-O-benzene sulfonate according to claim 16, wherein the poor solvent is normal hexane, petroleum ether, iso-propyl ether, iso-octane or a combination thereof.

19. The preparation method of paeoniflorin-6′-O-benzene sulfonate according to claim 16, wherein a volume-to-mass ratio of the good solvent to paeoniflorin-6′-O-benzene sulfonate obtained in step S3 is 8:1 mL/g to 16:1 mL/g.

20. The preparation method of paeoniflorin-6′-O-benzene sulfonate according to claim 16, wherein a volume-to-mass ratio of the poor solvent to paeoniflorin-6′-O-benzene sulfonate obtained in step S3 is 5:1 mL/g to 8:1 mL/g.
